(19) United States
(12) Patent Application Publication
(10) Pub. No.: US 2025/0340525 A1
(43) Pub. Date: Nov. 6, 2025

(54) FLUORINATED DIOXOLANE COMPOUND

(71) Applicant: EXPANESTHETICS, INC., Davis, CA (US)

(72) Inventors: Shane AUSTIN, Davis, CA (US); Frank WATERS, Alachua, FL (US); Wei XU, Alachua, FL (US)

(21) Appl. No.: 18/709,247

(22) PCT Filed: Dec. 6, 2023
(86) PCT No.: PCT/US2023/082681

§ 371 Date: May 30, 2024

Related U.S. Application Data
(60) Provisional application No. 63/431,630, filed on Dec. 9, 2022.

(51) Int. Cl. C07D 317/42

(52) U.S. Cl. CPC C07D 317/42

(57) ABSTRACT

A compound, 2,4,5-trifluoro-2-(trifluoromethyl)-1,3-dioxolane, useful as an anesthetic, solvent, as co-monomer in a polyacetals, or as a dispersant of fluorinated materials. The compound is synthesized as a mixture of cis- and trans-isomers, from which trans-2,4,5-trifluoro-2-(trifluoromethyl)-1,3-dioxolane can be isolated from the cis-isomers using distillation. The product mixture and the trans-isomer have independently been shown to cause loss-of-righting-reflex (LORR) in mice, indicating the dioxolanes' anesthetic activity.

### FIELD OF THE INVENTION

The present invention relates to a fluorinated dioxolane compound, and a method of synthesizing the same.

### BACKGROUND OF THE INVENTION

Halogenated dioxolane compounds have previously been identified as either having anesthetic activity, or as candidates to have anesthetic activity (see e.g., U.S. Pat. Nos. 3,749,791; International Patent Publication WO/2014/011235A1; Eger et al., Anesth. Analg., Vol. 60, No. 4, April 1981, pp. 201-203; and Bagnall et al., Journal of Fluorine Chemistry, 1977, Vol. 9, pp. 359-375, the disclosures of which are hereby incorporated by reference in their entireties). Each of these compounds had previously been registered with the Chemical Abstracts Service (CAS), and generally have been previously synthesized and characterized for their chemical properties. However, the number of halogenated dioxolane compounds is neither limited to those described in the references above nor by the CAS Registry itself, and instead encompasses an entire universe of chemically-viable compounds that have not been identified previously.

In general, a dioxolane is a heterocyclic acetal with the chemical formula (CH₂)₂O₂CH₂, related to tetrahydrofuran by interchange of one oxygen in place of a CH₂ group. 1,3-dioxolane compounds, such as TTD, have been previously identified to be useful as anesthetics, as solvents, as co-monomers in polyacetals, and as dispersants for fluorinated materials. Non-limiting examples of halogenated 1,3-dioxolanes identified to be useful as anesthetics are fluorine or chlorine-substituted 2,2-bis(trifluoromethyl)-1,3-dioxolanes (see U.S. Pat. No. 3,749,791, above) and 4,5-dichloro-2,2-difluoro-1,3-dioxolane (also referred to as “dioxychlorane”, see Eger, above). Non-limiting examples of additional halogenated dioxolanes identified as potentially having anesthetic activity and registered in the CAS registry (see e.g., International Patent Publication WO/2014/011235A1, above) are: 1,3-Dioxolane, 2,4,4,5-tetrafluoro-5-(trifluoromethyl)-(CAS No: 344303-08-8); 1,3-Dioxolane, 2-chloro-4,4,5-trifluoro-5-(trifluoromethyl)-(CAS No: 344303-05-5); 1,3-Dioxolane, 4,4,5,5-tetrafluoro-2-(trifluoromethyl)-(CAS No: 269716-57-6); 1,3-Dioxolane, 4-chloro-2,2,4-trifluoro-5-(trifluoromethyl)-(CAS No: 238754-29-5); 1,3-Dioxolane, 4,5-dichloro-2,2,4,5-tetrafluoro-, trans-(9Cl) (CAS No: 162970-78-7); 1,3-Dioxolane, 4,5-dichloro-2,2,4,5-tetrafluoro-, cis-(9Cl) (CAS No: 162970-76-5); 1,3-Dioxolane, 4-chloro-2,2,4,5,5-pentafluoro-(CAS No: 139139-68-7); 1,3-Dioxolane, 4,5-dichloro-2,2,4,5-tetrafluoro-(CAS No: 87075-00-1); 1,3-Dioxolane, 2,4,4,5-tetrafluoro-5-(trifluoromethyl)-, trans (9Cl) (CAS No: 85036-66-4); 1,3-Dioxolane, 2,4,4,5-tetrafluoro-5-(trifluoromethyl)-, cis-(9Cl) (CAS No: 85036-65-3); 1,3-Dioxolane, 2-chloro-4,4,5-trifluoro-5-(trifluoromethyl)-, trans (9Cl) (CAS No: 85036-60-8); 1,3-Dioxolane, 2-chloro-4,4,5-trifluoro-5-(trifluoromethyl)-, cis-(9Cl) (CAS No: 85036-57-3); 1,3-Dioxolane, 2,2-dichloro-4,4,5,5-tetrafluoro-(CAS No: 85036-55-1); 1,3-Dioxolane, 4,4,5-trifluoro-5-(trifluoromethyl)-(CAS No: 76492-99-4); 1,3-Dioxolane, 4,4-difluoro-2,2-bis(trifluoromethyl)-(CAS No: 64499-86-1); 1,3-Dioxolane, 4,5-difluoro-2,2-bis(trifluoromethyl)-, cis (9Cl) (CAS No: 64499-85-0); 1,3-Dioxolane, 4,5-difluoro-2,2-bis(trifluoromethyl)-, trans-(9Cl) (CAS No: 64499-66-7); 1,3-Dioxolane, 4,4,5-trifluoro-2,2-bis(trifluoromethyl)-(CAS No: 64499-65-6); 1,3-Dioxolane, 2,4,4,5,5-pentafluoro-2-(trifluoromethyl)-(CAS No: 55135-01-8); 1,3-Dioxolane, 2,2,4,4,5,5-hexafluoro-(CAS No: 21297-65-4); and 1,3-Dioxolane, 2,2,4,4,5-pentafluoro-5-(trifluoromethyl)-(CAS No: 19701-22-5).

Consequently, there exists a need to identify, synthesize, and characterize additional halogenated dioxolane compounds that can be analyzed for their potential anesthetic effects.

### SUMMARY OF THE INVENTION

The present invention provides a compound, 2,4,5-trifluoro-2-(trifluoromethyl)-1,3-dioxolane (TTD), which is represented by the general structure of Formula I, below:

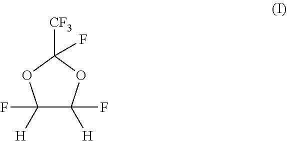 [SMILES: FC1OC(F)(C(F)(F)F)OC1F]

In some embodiments, TTD can be synthesized as a mixture of one or more stereoisomers, particularly as one or more enantiomers, one or more diastereomers, one or more epimers, one or more conformers, and any combination thereof, based on the stereocenters at the C₂, C₄, and C₅ positions. In some embodiments, the stereoisomer mixture can comprise TTD isomers in which the C₄ and C₅ fluorine atoms are oriented in opposing directions (hereinafter, “trans-TTD”). In some embodiments, the stereoisomer mixture can comprise TTD isomers in which the C₄ and C₅ fluorine atoms are oriented in the same direction (hereinafter, “cis-TTD”). In some embodiments, cis-TTD can further be identified by their orientation relative to the trifluoromethyl moiety at C₂, as either cis-syn or cis-anti isomers. In some embodiments, a mixture of TTD isomers can be separated, including distilled, to isolate trans-TTD isomers from cis-TTD isomers.

In some embodiments, the compound of Formula I can be combined with one or more additional compounds to form a composition. In some embodiments, the compound of Formula I can be combined with one or more additional anesthetic compounds to form a mixture.

In some embodiments, any one or a mixture of the TTD isomers can exhibit anesthetic activity. In some embodiments, a trans-isomer of TTD has anesthetic activity. In some embodiments, a cis-isomer of TTD has anesthetic activity. In some embodiments, a composition comprising a mixture of a cis-isomer of TTD and a trans-isomer of TTD has anesthetic activity.

### DETAILED DESCRIPTION OF THE INVENTION

### Definitions

As used herein, the term “composition” refers to a mixture of the compound of Formula I with one or more additional compounds.

### Embodiments of the Present Invention

As indicated above, the present invention encompasses both an isolated form of the compound, 2,4,5-trifluoro-2-(trifluoromethyl)-1,3-dioxolane (TTD), compositions comprising two or more stereoisomers of TTD, and compositions comprising one or more TTD stereoisomers and one or more additional compounds. Non-limiting examples of types of additional compounds are a salt, liquid or solid filler, stabilizer, dispersing agent, suspending agent, diluent, excipient, thickening agent, solvent, encapsulating material, coating, antibacterial agent, antifungal agent, or absorption delaying agent, and any mixture or combination thereof. In some embodiments, TTD and/or any of its stereoisomers may be isolated or present within a composition as, or converted into, a salt, such as in combination with an acceptable cation or anion, as is well known in the art.

In various embodiments, an amount of TTD and any additional compound in a composition can be selected to any desired concentration. By way of example, the composition may comprise between 0.1% and 100% by weight TTD.

In various embodiments, non-limiting examples of an additional compound are a sugar, such as lactose, glucose and sucrose; a starch, such as corn starch or potato starch; a cellulose, and its derivatives, such as sodium carboxymethyl cellulose, ethyl cellulose, or cellulose acetate, or a mixture thereof; a powdered tragacanth; a malt; a gelatin; a talc; an excipient, for example cocoa butter or suppository wax; an oil, for example peanut oil, cottonseed oil, safflower oil, sesame oil, olive oil, corn oil and soybean oil; a glycol, for example propylene glycol; a polyol, for example glycerin, sorbitol, mannitol, polyethylene glycol, or a mixture thereof; an esters, for example ethyl oleate and ethyl laurate; agar; a buffering agents, for example magnesium hydroxide or aluminum hydroxide; a surface active agent; alginic acid; pyrogen-free water; isotonic saline; Ringer's solution; ethyl alcohol; and a phosphate buffer solution; and any mixture thereof.

### EXAMPLES

The following examples illustrate embodiments of the invention. However, it is to be understood that the following are only exemplary or illustrative of the present invention, and are not intended to limit the invention in any way, and numerous modifications and alternative compositions, methods, and systems may be devised by those skilled in the art without departing from the spirit and scope of the present invention.

### Example 1: Synthesis of Trans-Isomers of TTD

Generally, the synthesis of TTD comprised the following reaction steps: (a) the synthesis of 2-(trifluoromethyl)dioxolane (Intermediate 1) upon combining trifluoroacetaldehyde methylhemiacetal and 2-bromoethanol; (b) UV-catalyzed chlorination of Intermediate 1 to form 2,4,4,5,5-pentachloro2-trifluoromethyl-1,3-dioxolane (Intermediate 2); (c) selective fluorination of Intermediate 2 to form 4,5,-dichloro-2,4,5-trifluoro-2-trifluoromethyl-1,3-dioxolane (Intermediate 3); (d) dechlorination of Intermediate 3 with tributylin hydride to form a TTD mixture comprising cis- and trans-isomers of TTD (Product); and (c) distillation of Compound 4 to isolate trans-isomers of TTD from cis-isomers of TTD.

### Step 1: Formation of 2-(trifluoromethyl)-dioxolane

2614 g (15 mol) of potassium carbonate and 3.2 L of dimethylformamide (DMF) were charged in a 12 L flask equipped with heating mantle, mechanical stirrer, thermometer, and a distillation head and water condenser vented to a trap then to a gas bubbler.

A mixture of 1.44 kg of trifluoroacetaldehyde methylhemiacetal (10 mol) and 2 kg of 2-bromoethanol (16 mol) was added slowly to the flask at room temperature with good stirring. The reaction was exothermic, and the reaction temperature increased to 62° C. during addition (3 hours and 15 minutes). Gas flow was observed during addition.

The mixture was stirred for 2 hours without heating, during which time the reaction progress was monitored by GC. After 2 hours, heat was applied, and the reaction temperature was slowly increased to 100° C. At 100° C., gentle refluxing was observed, and some liquid was collected in the trap. Product take-off was begun and continued until the head temperature reached 130° C. and pot temperature reached 150° C. Collection time was around 4 hours.

The crude product was washed twice with 2 liters (L) water. After separating the organic layer, the remaining product was dried over magnesium sulfate and filtered to give 920 grams of 2-(trifluoromethyl)-dioxolane (yield, 65%, purity 97%). The filtered 2-(trifluoromethyl)dioxolane was used for the next step directly without further purification.

### Step 2: Chlorination of the 2-(trifluoromethyl)-dioxolane product of Reaction 1 to form 2,4,4,5,5-pentachloro2-trifluoromethyl-1,3-dioxolane

A 2 L flask was equipped with a dry ice-acetone condenser, gas inlet, heating mantle and thermometer. Chlorine gas was dispersed into the flask through the gas inlet and the 2-(trifluoromethyl)-dioxolane product of Step 1 was illuminated with a sunlight lamp. Initially, the reaction temperature was maintained under 80° C., but was then increased to 100° C. after partial chlorination product was formed. The reaction was determined to be complete after 48 hours, using gas chromatography. Excess chlorine was removed from the reaction mixture upon flushing the reaction mixture with nitrogen. The crude product was flash distilled to obtain 1850 grams of 2,4,4,5,5-pentachloro-2-trifluoromethyl-1,3-dioxolane as a colorless liquid, which was collected at 56-60° C./15 torr with a yield of 91%. Alternatively, a UV light can be used to initiate chlorination, and the reaction can be completed in less than 24 hours.

### Step 3: Selective Substitution of Fluorine Into the 2,4,4,5,5-pentachloro2-trifluoromethyl-1,3-dioxolane of Reaction 2 to Form 4,5-dichloro-2,4,5-trifluoro-2-trifluoromethyl-1,3-dioxolane

A 12 L flask was equipped with mechanical stirrer, heating mantle, distillation head with water condenser, and an addition funnel. The system was dried by heating with a heat gun and maintained under a nitrogen atmosphere. Five (5) kg (28 mol) of antimony trifluoride (freshly dried and crushed), and 5 kg (15.9 mol) of 2,4,4,5,5-pentachloro-2-trifluoromethyl-1,3-dioxolane made according to the methods of Step 1 and Step 2 were added to the flask and heated slowly to 40° C. with stirring. 200 mL of antimony pentachloride was added to the mixture at a rate of 5 mL/30 min, resulting in an exothermic reaction. Without being limited by a particular theory, it is believed that drying and crushing the antimony trifluoride facilitated its complete reaction with 2,4,4,5,5-pentachloro-2-trifluoromethyl-1,3-dioxolane. Gentle refluxing was observed when the reaction temperature reached 100° C. The temperature at the distillation head and flask were used to monitor the progress. When head temperature reached 74° C. and reaction temperature stayed at 78° C., the fluorination product was collected. Around 4 kg of crude material was collected at 74-78° C. The crude product (4,5-dichloro-2,4,5-trifluoro-2-trifluoromethyl-1,3-dioxolane) was then stirred with 500 mL of concentrated hydrochloric acid for 15 minutes. The organic layer was separated and washed with water then dried over magnesium sulfate and filtered. The filtered product was then distilled using a 3-foot-glass packed column to obtain 2885 g of purified 4,5-dichloro-2,4,5-trifluoro-2-trifluoromethyl-1,3-dioxolane, which was obtained at 70° C. as a mixture of cis- and trans-isomers (Purity: 99.5%; yield: 68%).

### Step 4: Synthesis of trans-2,4,5-trifluoro-2-trifluoromethyl-1,3-dioxolane

A 22 L flask was equipped with mechanical stirrer, distillation head with water condenser, heating mantle, sunlight lamp, and addition funnel. The flask was charged with 10 kg of tributyltin hydride and heated to 85° C. before adding dropwise 2885 g (10.9 mol) of the 4,5-dichloro-2,4,5-trifluoro-2-trifluoromethyl-1,3-dioxolane product of Step 3. Because the reaction was exothermic, the heating mantle was turned off once the reaction was initiated. The reaction temperature was kept at ˜100° C. by controlling the rate of addition of 4,5-dichloro-2,4,5-trifluoro-2-trifluoromethyl-1,3-dioxolane. Once the addition was complete, the mixture was stirred at 100° C. overnight. The reaction progress was monitored by gas chromatography. The final 2,4,5-trifluoro-2-trifluoromethyl-1,3-dioxolane (TTD) product, containing both cis- and trans-isomers and minimal impurities, was distilled from the flask, using reduced pressure at the end of the distillation to recover additional material from the reaction mixture. Without being limited by a particular theory, it is believed that the significant excess of the tributyltin hydride in the reaction facilitated the nearly complete reduction of the 4,5-dichloro-2,4,5-trifluoro-2-trifluoromethyl-1,3-dioxolane into the final TTD product.

### Step 5: Separation of the Trans-Isomer of TTD from the Cis-Isomers of TTD

The mixture of cis- and trans-isomers of TTD produced in Step 5, above, was fractionally distilled using a 3-foot-glass packed column. 892 g of the trans-isomer of TTD was collected at 64° C., (purity 99.6%, yield 42%), while 525g of mixed cis-isomers was collected at (93° C., purity, 99.0%; yield, 25%).

### Example 2: Characterization of the Trans-Isomer of TTD by GC/MS

0.2 μL of the product of Step 5 above was injected into a Zebron™ ZB-5MSplus GC Column coupled to an Agilent 5977A Series single quadrupole Gas Chromatography/Mass Spectrometry device (GC/MSD). The resulting mass spectrum is illustrated below.

The mass spectrum indicates a visible peak at 177 m/z, corresponding to the expected molecular weight of trans-isomer of TTD (196 amu) with the loss of a single fluorine atom (19 amu). Without being limited by a particular theory, it is believed that the loss of the fluorine atom occurred as a result of ionizing the TTD to facilitate the mass spectrometry experiment. Therefore, it is believed that trans-isomer of TTD was present within the original sample.

### Example 3: Characterization of the TTD Isomer Mixture by F¹⁹-NMR

A sample of the product mixture from Step 4 was prepared for NMR spectroscopy by dissolving the mixture into deuterated chloroform. F¹⁹-NMR spectra were collected on a Bruker Avance HDIII NMR Spectrometer operating at 600 MHz equipped with a 5 mm smart probe. Peaks on the resulting NMR spectra were of sufficient quality to be assigned to respective H¹ and F₁₉ atoms within each of the compounds in the sample. Assignment tables for the trans-isomer of TTD (Compound 1) and cis-isomers of TTD (Compounds 2 and 3) are presented below.

TABLE 1
Compound 1

| | delta | H1 | H2 | F1 | F2 | F4 | F6 |
|---|---|---|---|---|---|---|---|
| F1 | −131.38 | 60.5 | 4.0 | X | 4.1 | 20.5 | 4.1 |
| F2 | −131.93 | 3.5 | 58.5 | 4.1 | X | 10.1 | 0.0 |
| F4 | −84.52 | 0.0 | 4.5 | 10.1 | 20.5 | X | 0.0 |
| F6 | −85.59 | 0.0 | 0.0 | 4.2 | 0.0 | 0.0 | X |

TABLE 2
Compound 2

| | delta | H1 | H2 | F1 | F2 | F4 | F6 |
|---|---|---|---|---|---|---|---|
| F1 | −143.16 | ca. 68 | ca. 10 | X | — | 15.1 | 1.8 |
| F2 | −143.16 | ca. 10 | ca. 68 | — | X | 15.1 | 1.8 |
| F4 | −85.78 | 0.0 | 0.0 | 15.5 | 15.5 | X | 0.0 |
| F6 | −85.66 | 0.0 | 0.0 | 1.8 | 1.8 | 0.0 | X |

TABLE 3
Compound 3

| | delta | H1 | H2 | F1 | F2 | F4 | F6 |
|---|---|---|---|---|---|---|---|
| F1 | −140.87 | ca. 67 | ca. 9 | X | — | 6.5 | 0.0 |
| F2 | −140.87 | ca. 9 | ca. 67 | — | X | 6.5 | 0.0 |
| F4 | −84.46 | 5.9 | 5.9 | 6.5 | 6.5 | X | 0.0 |
| F6 | −85.83 | 0.0 | 0.0 | 0.0 | 0.0 | 1.6 | X |

In Tables 1-3 above, Compound 1 can be identified as being the only isomer in which the CHF groups are different, as the fluorine atom of one of the CHF groups couples with the trifluoromethyl group (F6 in Tables 1-3). Without being limited by a particular theory, it is believed that this is a through-space coupling, since the coupling otherwise would be over 5 bonds.

In Compound 1, the coupling of the CF₃ fluorine atoms (F6) with the fluorine atoms of the CHF groups five bonds away are 4.1 Hz with the cis (F1) and 0 Hz with the trans (F2)-⁵J_{F1-F6}=4.1 Hz and ⁵J_{F2-F6}=0.0 Hz. The couplings of F4 are 20.5 Hz with the trans (F1) and 10.1 Hz with the cis (F2).

Of the two symmetric cis isomers, Compound 2, with a molar ratio to 1 of 0.34:1, has the larger ⁵J_{F1,F2-F4}=15.1 Hz and also displays a coupling F1,F2-F6. Accordingly, it was assigned as the isomer in which F1,F2 are cis to CF₃.

### Example 4: Anesthetic Activity of TTD

A sample of the product mixture formed in Step 4 of the synthesis of Example 1, comprising both cis- and trans-isomers of TTD (the “TTD mixture”), was administered to mice via inhalation exposure in a microcircuit connected using unidirectional airflow valves to a CO₂ absorbent chamber and a glass syringe. A small amount of liquid TTD mixture was transferred into the glass syringe where it volatilized. Mice were transferred to the small cylindrical chamber, and the glass syringe was connected to the microcircuit in series. The syringe was then slowly pumped to circulate the vaporized TTD mixture/air mixture through the microcircuit, until the mice indicated a loss-of-righting-reflex (LORR). Accordingly, and without being limited by a particular theory, it is believed that the dose of the TTD mixture introduced into the microcircuit demonstrated an anesthetic potency. Delivery of TTD mixture was discontinued, and the mice were allowed to recover.

Separately, the trans-isomer of TTD isolated in step 5 of Example 1 above was administered to mice via inhalation exposure in a microcircuit connected using unidirectional airflow valves to a CO₂ absorbent chamber and a glass syringe. A small amount of the TTD was transferred into the glass syringe where it volatilized. Mice were transferred to the small cylindrical chamber, and the glass syringe was connected to the microcircuit in series. The syringe was then slowly pumped to circulate the vaporized TTD mixture/air mixture through the microcircuit, until the mice indicated a loss-of-righting-reflex (LORR). Without being limited by a particular theory, it is believed that the dose of the trans-isomer of TTD introduced into the circuit demonstrated an anesthetic potency. Delivery of the TTD was discontinued, and the mice were allowed to recover.

We claim:

1. A compound, 2,4,5-trifluoro-2-(trifluoromethyl)-1,3-dioxolane.

2. The compound of claim 1, selected from the group consisting of cis-2,4,5-trifluoro-2-(trifluoromethyl)-1,3-dioxolane, trans-2,4,5-trifluoro-2-(trifluoromethyl)-1,3-dioxolane, and a combination thereof.

3. The compound of claim 2, consisting of trans-2,4,5-trifluoro-2-(trifluoromethyl)-1,3-dioxolane.

4. The compound of claim 2, consisting of cis-2,4,5-trifluoro-2-(trifluoromethyl)-1,3-dioxolane.

5. The compound of claim 2, consisting of a mixture of cis-2,4,5-trifluoro-2-(trifluoromethyl)-1,3-dioxolane and trans-2,4,5-trifluoro-2-(trifluoromethyl)-1,3-dioxolane.

6. A composition comprising the compound of claim 1.
